(19) United States
(12) Patent Application Publication
(10) Pub. No.: US 2026/0165575 A1
(43) Pub. Date: Jun. 18, 2026

(54) MEDICAL ASSISTANCE DEVICE, MEDICAL ASSISTANCE METHOD, AND NON-TRANSITORY RECORDING MEDIUM

(71) Applicant: CASIO COMPUTER CO., LTD., Tokyo (JP)

(72) Inventor: Yukiko OKUDA, Tokyo (JP)

(21) Appl. No.: 19/423,666

(22) Filed: Dec. 17, 2025

(30) Foreign Application Priority Data
Dec. 18, 2024 (JP) 2024-221352

(51) Int. Cl. A61B 1/303; A61B 1/00; A61B 1/06

(52) U.S. Cl. CPC A61B 1/303; A61B 1/000095; A61B 1/00048; A61B 1/0655

(57) ABSTRACT

A medical assistance device that assists in performing colposcopy includes one or more processors to: identify, based on a plurality of images obtained by continuous image capturing, the continuous image capturing continuously capturing images of a target region to which acetic acid treatment is applied, a time at which a predetermined change associated with acetic acid treatment occurs in the target region or an image captured at the time; and perform, based on the identified time or the identified image, at least one of image capturing condition control in the continuous image capturing, image recording control in the continuous image capturing, and display control of an image recorded by the continuous image capturing.

### CROSS-REFERENCE TO RELATED APPLICATIONS

This application claims the benefit of Japanese Patent Application No. 2024-221352, filed on Dec. 18, 2024, the entire disclosure of which is incorporated by reference herein.

### FIELD OF THE INVENTION

This application relates generally to a medical assistance device, a medical assistance method, and a non-transitory recording medium.

### BACKGROUND OF THE INVENTION

As one of examinations of cervical cancer, colposcopy in which the uterine cervix is observed using a colposcope is performed. In the colposcopy, by applying acetic acid to the uterine cervix, the surface of the uterine cervix changes in color in a case where there is a lesion, which enables the lesion site to be observed. For example, Patent Literature 1 (Unexamined Japanese Patent Application Publication (Translation of PCT Application) No. 2010-535579) discloses a method for capturing images before and after acetic acid treatment is applied to a lesion and detecting a lesion region based on the captured images.

### SUMMARY OF THE INVENTION

A medical assistance device according to the present disclosure is a medical assistance device that assists in performing colposcopy and includes one or more processors to: identify, based on a plurality of images obtained by continuous image capturing, the continuous image capturing continuously capturing images of a target region to which acetic acid treatment is applied, a time at which a predetermined change associated with acetic acid treatment occurs in the target region or an image captured at the time; and perform, based on the identified time or the identified image, at least one of image capturing condition control in the continuous image capturing, image recording control in the continuous image capturing, and display control of an image recorded by the continuous image capturing.

### BRIEF DESCRIPTION OF DRAWINGS

A more complete understanding of this application can be obtained in a case where the following detailed description is considered in conjunction with the following drawings, in which:

FIG. 1 is a diagram illustrating an outline of an overall configuration of a medical assistance device according to Embodiment 1 of the present disclosure;

FIG. 2 is a flowchart illustrating a control process in an acetic acid treatment mode according to Embodiment 1 of the present disclosure;

FIG. 3A is a diagram illustrating a display of the medical assistance device;

FIG. 3B is a diagram illustrating a display screen in the acetic acid treatment mode;

FIG. 4 is another flowchart illustrating the control process in the acetic acid treatment mode according to Embodiment 1 of the present disclosure;

FIG. 5 is a flowchart illustrating a control process in an acetic acid treatment-dedicated reproduction mode according to Embodiment 1 of the present disclosure;

FIG. 6 is a flowchart illustrating a control process in a biopsy mode according to Embodiment 1 of the present disclosure;

FIG. 7 is a diagram illustrating a display screen in the biopsy mode;

FIG. 8 is a flowchart illustrating image extraction processing according to Embodiment 2 of the present disclosure;

FIG. 9 is another flowchart illustrating the image extraction processing according to Embodiment 2 of the present disclosure; and

FIG. 10 is still another flowchart illustrating the image extraction processing according to Embodiment 2 of the present disclosure.

### DETAILED DESCRIPTION OF THE INVENTION

In conventional colposcopy, consideration has not been given to reducing a burden on an operator or a diagnostician who performs, in addition to capturing an image of a lesion, various operations such as application of acetic acid, observation of a lesion, determination of necessity of biopsy, collection of cells or tissue for biopsy, recording of an operation status, and recording of diagnosis of a lesion. In addition to the case where acetic acid is applied to the uterine cervix, the same applies to a case where acetic acid or other chemical liquid is applied to another disease region, such as the ear, nose, and throat and the digestive tract, to facilitate observation of a state of a lesion. The present disclosure addresses the above-described problem and reduces a burden on the operator or the diagnostician who performs observation or diagnosis of a lesion. Furthermore, the present disclosure enhances the efficiency of data acquisition by eliminating unnecessary data and the reproducibility of observation. A medical assistance device according to Embodiment 1 of the present disclosure is applied to a colposcopy camera that is an image capturing device for examination of the vagina and the uterine cervix, and more generally to a close-up camera that is an image capturing device to capture an image of details of a subject at a close distance to the subject, or the like. In addition, the medical assistance device may be a system that is a combination of an image capturing device, such as a colposcopy camera, and a viewer device. In addition, the viewer device may be an information processing device such as a personal computer. Herein, the following description is made using a colposcopy camera as an example.

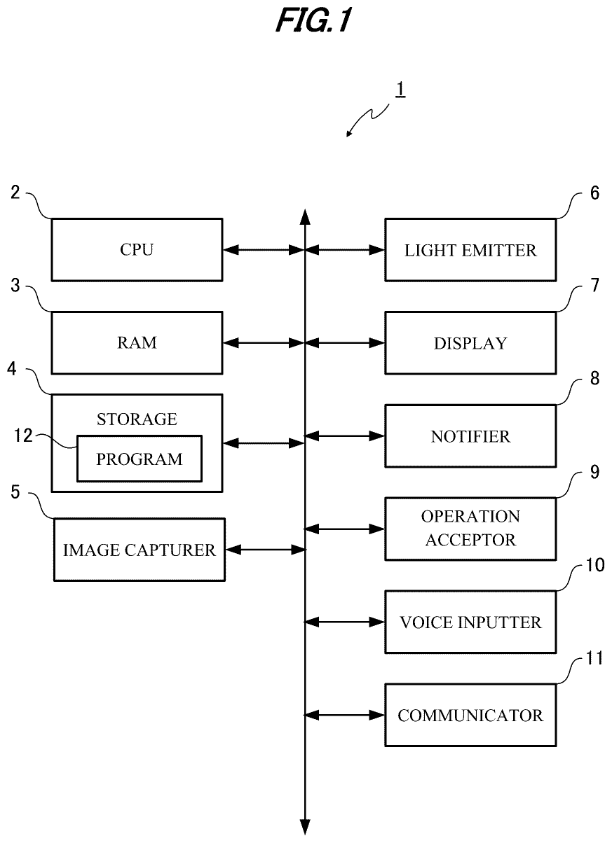

A medical assistance device 1 includes, as illustrated in FIG. 1, a central processing unit (CPU) 2, a random access memory (RAM) 3, a storage 4, an image capturer 5, a light emitter 6, a display 7, a notifier 8, an operation acceptor 9, a voice inputter 10, and a communicator 11, and the foregoing constitutional elements are interconnected by a bus.

The CPU 2 is a processor that controls operation of the medical assistance device 1 by retrieving and executing a program 12 stored in the storage 4 and performing various types of operation processing. Note that the medical assistance device 1 may have a plurality of processors (for example, a plurality of CPUs 2), and a plurality of processes executed by the CPU 2 of the present embodiment may be executed by the plurality of processors. At the time of capturing an image of the uterine cervix, the CPU 2 causes the light emitter 6 to emit light in accordance with a setting or operation by the operator or the diagnostician (hereinafter referred to as an operator or the like), causes the image capturer 5 to perform image capturing in accordance with operation by the operator or the like who instructs image capturing, and thereby generates image data of medical images obtained by capturing the uterine cervix. The RAM 3 functions as a work area for the CPU 2 and temporarily stores the program 12 and data. The image capturer 5 includes an optical system that causes incident light to form an image, an image sensor that detects incident light caused to form an image by the optical system, an image processor that generates image data, based on a detection signal output from the image sensor, and the like. The optical system may include, for example, a mirror, a lens group, and the like. As the image sensor, for example, a charge coupled device (CCD), a complementary metal oxide semiconductor (CMOS), or the like can be used. The image processor performs amplification and A/D conversion of a detection signal from the image sensor, and generates image data of a captured subject. In addition, the image processor may perform various types of image processing on the generated image data.

The light emitter 6 radiates illuminating light to illuminate the subject in a direction in which the image capturer 5 captures an image of the subject. As described above, the light emitter 6 includes a normal light emitting light, a green light emitting light, and a polarized light emitting light. Turning on and off of light emission by the light emitter 6 is controlled by a control signal transmitted from the CPU 2. The display 7 includes a display device such as a liquid crystal display. The display 7 displays a captured image as a live view image in real time, reproduces and displays a captured image, or displays various types of operation icons, on a display screen of the display device, in accordance with the image data and a control signal transmitted from the CPU 2. The notifier 8 includes a speaker that emits a notification sound. The notifier 8, as described later, notifies the operator or the like by emitting a detection sound in a case where a foreign object is detected in an image displayed on the display 7, in a case where there is a change in color tone in the image, or the like. The operation acceptor 9 includes a touch panel that is provided overlaid on the display screen and an operation button or the like provided on a case of the camera. The operation acceptor 9 detects contact of a finger or the like with the touch panel or an operation on the operation button and outputs an operation signal corresponding to a detection result to the CPU 2. The voice inputter 10 includes a microphone to input a voice. For example, in a case where a voice is input to the microphone by the operator or the like uttering specific words, the voice inputter 10 outputs an operation signal corresponding to the words to the CPU 2. The communicator 11 includes a communication module such as a LAN and performs data communication with a medical image management device or the like via a network.

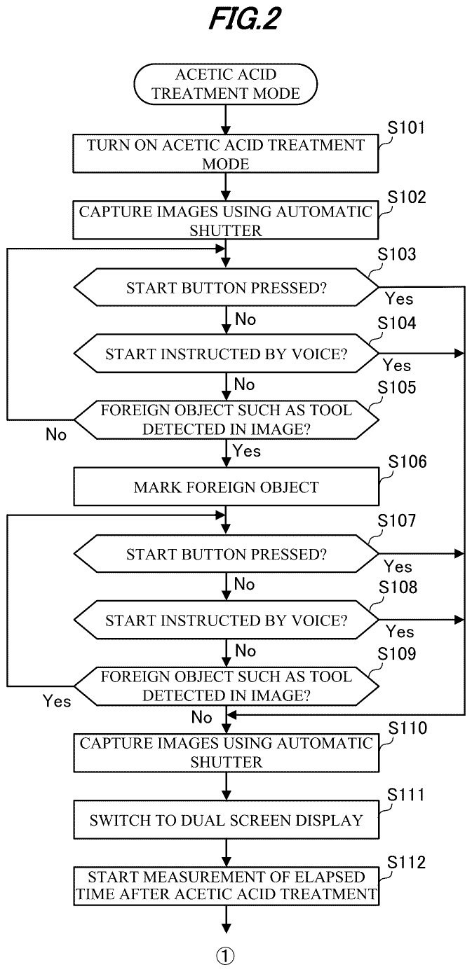

The medical assistance device 1 has functions of an acetic acid treatment mode to assist the operator or the like in performing acetic acid treatment processing of applying acetic acid to the uterine cervix and observing a lesion site in the colposcopy, an acetic acid treatment-dedicated reproduction mode to assist the operator or the like in extraction of an image indicating a most robust lesion region in the acetic acid treatment, and a biopsy mode to assist the operator or the like in biopsy to collect tissue from a lesion site detected by the acetic acid treatment processing. The above-described modes are described in FIG. 2 and subsequent drawings.

FIG. 2 is a flowchart illustrating a control process executed by the CPU 2 in the acetic acid treatment mode. In a case where the medical assistance device 1 is placed at a position where the image capturer 5 is able to capture an image of a target region of a patient who is a subject, the operator or the like first sets the camera to the “acetic acid treatment mode” (step S101). In this processing, the “acetic acid treatment mode” is turned on by pressing a button on a camera body or by touching the screen on the display device, and an acetic acid treatment mode screen is displayed.

In a case where the camera transitions to the “acetic acid treatment mode”, the image capturer 5 performs image capturing, using an automatic shutter (step S102). At the time of image capturing, the light emitter 6 activates the normal light emitting light, the green light emitting light, and the polarized light emitting light in a sequential manner and irradiates the target region of the patient, who is the subject, with normal light, green light, and polarized light as the illuminating light. The image capturer 5 performs image capturing every time each of the normal light, the green light, and the polarized light is radiated. As used herein, the image capturing refers to selectively recording an image captured at the time of an automatic release of the shutter or the like from a live view image in which continuously captured images are displayed in real time. Through this processing, images of the target region of the patient before the acetic acid treatment are captured.

After the image capturing is performed, the operator or the like applies acetic acid to the target region of the patient by pressing a swab soaked in, for example, 3 to 5% concentration acetic acid solution for 10 to 15 seconds on the target region. In a case where the application of acetic acid is finished, assistance in the observation of the target region after the acetic acid treatment is started by the operator or the like operating a start button. The start button is provided on the camera body, and the CPU 2 determines whether or not the start button has been pressed (step S103). In a case where the CPU 2 determines that the start button is pressed (step S103: Yes), the CPU 2 starts the assistance in the observation of the target region after the acetic acid treatment in and after step S110. Note that the start button may be a button displayed on the display screen of the display 7 and by touching the start button displayed on the screen, the assistance in the observation of the target region after the acetic acid treatment is started.

In contrast, in a case where the start button has not been pressed and the CPU 2 does not determine that the start button is pressed (step S103: No), the CPU 2 next determines whether or not a start input by voice has been made (step S104). In a case where the operator or the like utters a specific word, such as “start”, the voice inputter 10 detects the utterance and outputs an operation signal corresponding to the detection result to the CPU 2. In a case where the CPU 2 receives the output from the voice inputter 10 and determines that the start input by voice is made (step S104: Yes), the CPU 2 starts the assistance in the observation of the target region after the acetic acid treatment in and after step S110. Because of this configuration, the operator or the like can cause the assistance in the observation of the target region after the acetic acid treatment to be started at a time that the operator or the like himself/herself determines, without touching the camera with a hand.

In contrast, in a case where the word “start” has not been uttered and the CPU 2 does not determine that the start input by voice is made (step S104: No), the CPU 2 next starts, based on detecting termination of the acetic acid treatment processing from an image of the target region input by the image capturer 5, the assistance in the observation of the target region after the acetic acid treatment in and after step S110. The image that is input from the image capturer 5 in this processing is a live view image obtained by continuously capturing images input from the image capturer 5. First, the CPU 2 performs image processing on an image of the target region input from the image capturer 5 and determines whether or not a foreign object other than the target region is detected (step S105). The image processor of the image capturer 5 detects a foreign object by performing color extraction from the image of the target region, performing edge detection with respect to an extracted color region, and extracting a contour shape that is geometric information of the color region. As used herein, the foreign object refers to a tool such as forceps, tweezers, a swab, and a syringe, or the like that is different from the uterine cervix, which is the target region. In a case where such a foreign object is captured in the image, it is indicated that the target region is being subjected to the acetic acid treatment processing. In contrast, in a case where no foreign object is captured in the image, it is indicated that the process is in a state before or after the acetic acid treatment processing is performed on the target region.

In a case where in step S105, it is determined that a foreign object is detected (step S105: Yes), the CPU 2 marks a portion where the foreign object is detected in such a manner as to enclose the portion and displays the marked portion on the screen of the display device by a dotted line or the like (step S106). In this example, since a foreign object is detected, the target region is being subjected to the acetic acid treatment processing. In addition, in a case where a foreign object is detected, a notification sound indicating the detection of a foreign object, such as a sound “bleep, bleep”, is emitted from the notifier 8 and notifies the operator or the like of the detection of a foreign object.

In contrast, in a case where in step S105, it is not determined that a foreign object is detected (step S105: No), it is determined that the process is in a state before the acetic acid treatment processing is performed on the target region, the process returns to step S103, and the processing in steps S103 to S105 is repeated until a foreign object is detected. Note, however, that in a case where while the processing in steps S103 to S105 is repeated, the start button is pressed or a start input by voice is made, the process proceeds to start processing of assistance in the observation of the target region after the acetic acid treatment in and after step S110. Through this processing, in a case where the acetic acid treatment processing is terminated even without detecting a foreign object, the operator or the like can start the assistance processing in the observation of the target region after the acetic acid treatment.

Returning to step S106, in a case where the detected foreign object is marked, the CPU 2, as with step S103, determines whether or not the start button has been pressed (step S107). In this processing, the operator or the like can press the start button at the time that the operator or the like himself/herself determines, while confirming the marked portion on the screen of the display device. In a case where the CPU 2 determines that the start button is pressed (step S107: Yes), the CPU 2 starts the assistance in the observation of the target region after the acetic acid treatment in and after step S110.

In contrast, in a case where the start button has not been pressed and the CPU 2 does not determine that the start button is pressed (step S107: No), the CPU 2 next determines whether or not a start input by voice has been made, as with step S104 (step S108). In a case where the CPU 2 receives an output from the voice inputter 10 and determines that a start input by voice is made (step S108: Yes), the CPU 2 starts the assistance in the observation of the target region after the acetic acid treatment in and after step S110. Because of this configuration, the operator or the like can cause the assistance in the observation of the target region after the acetic acid treatment to be started at a time that the operator or the like himself/herself determines, without touching the camera with a hand.

In contrast, in a case where the CPU 2 does not determine that a start input by voice is made (step S108: No), the CPU 2 determines whether or not the foreign object other than the target region is detected in an image of the target region input from the image capturer 5 (step S109). In a case where it is not determined that the foreign object is detected (step S109: No), that is, in a case where the mark on the screen of the display device disappears and the foreign object vanishes from the screen, the CPU 2 determines that the acetic acid treatment processing on the target region is terminated and starts the assistance in the observation of the target region after the acetic acid treatment in and after step S110. In addition, in a case where the foreign object becomes undetectable, a notification sound indicating that the foreign object is not detected, such as a sound “bleep”, is emitted from the notifier 8 and notifies the operator or the like that the foreign object disappeared from the screen and the acetic acid treatment processing is terminated.

In contrast, in a case where in step S109, the CPU 2 determines that the foreign object is detected (step S109: Yes), the process returns to step S107, and the processing in steps S107 to S109 is repeated until the foreign object having disappeared from the screen is detected. Note, however, that in a case where while the processing in steps S107 to S109 is repeated, the start button is pressed or a start input by voice is made, the process proceeds to the start processing of the assistance in the observation of the target region after the acetic acid treatment in and after step S110. Because of this configuration, even in a case where the acetic acid treatment processing is terminated while the foreign object is mistakenly not detected to have disappeared, the operator or the like can start the assistance processing in the observation of the target region after the acetic acid treatment.

By applying acetic acid to the uterine cervix, in a case where there is a lesion, the epithelium changes whiter than the normal epithelium in surroundings. In addition, a yellow color tone is sometimes observed as a change in color. Hereinafter, the above-described reaction that changes the color to white (yellow) is referred to as a white reaction. In step S110, the image capturer 5 performs image capturing, using the automatic shutter. At the time of image capturing, as image capturing condition control, the light emitter 6, differing from step S102, does not activate the green light emitting light but activates the normal light emitting light and the polarized light emitting light in a sequential manner and irradiates the target region of the patient with the normal light and the polarized light. The image capturer 5 performs image capturing every time each of the normal light and the polarized light is radiated. Because of this configuration, the image capturer 5 captures images of the target region before the white reaction.

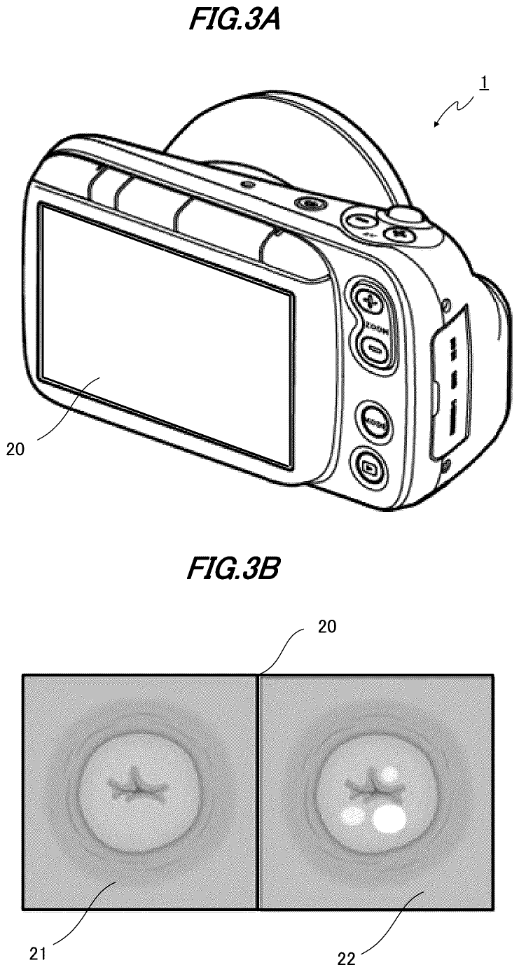

After the images of the target region before the white reaction are captured, the display 7 switches the display screen of the display device from a single screen display that displays only a live view image input from the image capturer 5 to a dual screen display that is obtained by dividing the screen into halves and adding a captured image of the target region before the white reaction (step S111). In FIGS. 3A and 3B, an example of the screen of the display device on which dual screens are displayed is illustrated. FIG. 3A illustrates a screen 20 of the medical assistance device 1, and FIG. 3B illustrates a state in which dual screens are displayed. As illustrated in FIG. 3B, the screen is divided into halves on the right and left sides, and an image of the target region before the white reaction that is automatically captured is displayed on a left-hand side screen 21 and a live view image during the white reaction is displayed on a right-hand side screen 22. By displaying an image before the white reaction and a live view image during the white reaction side by side, the operator or the like can, while confirming the target region before the reaction, compare and confirm a real-time change.

After the screen is switched to the dual screen display, the CPU 2 starts measuring elapsed time after the acetic acid treatment (step S112). In a case where the measurement of time is started, the elapsed time is displayed on the screen 20 of the display device. Because of this configuration, the operator or the like can check the elapsed time while observing the target region.

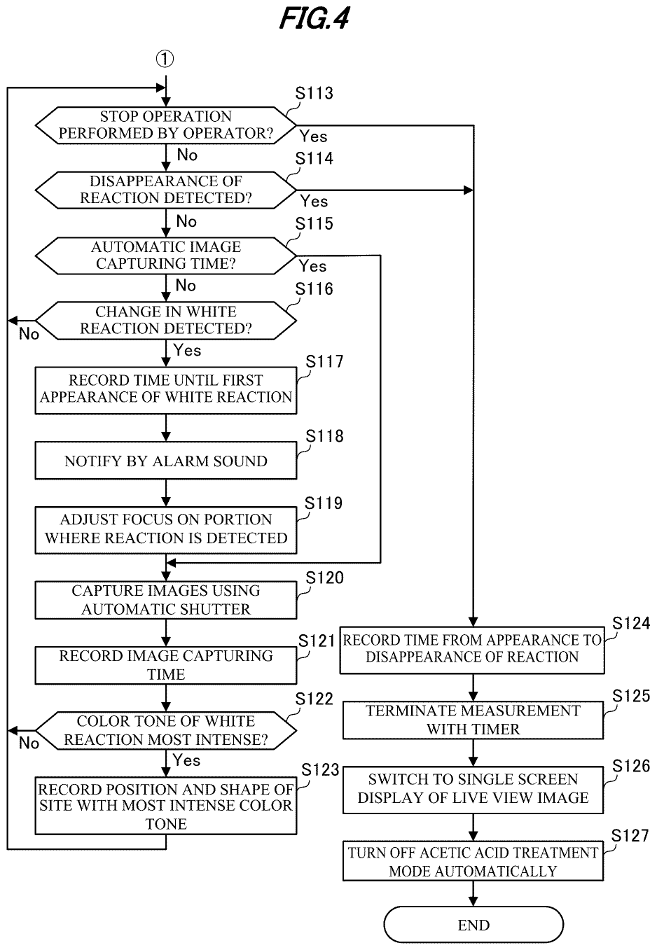

After the measurement of time after the acetic acid treatment is started, the CPU 2 determines whether or not a stop operation to terminate the measurement has been performed by the operator or the like, in step S113 in FIG. 4. In a case where the stop operation has not been performed (step S113: No), the CPU 2 determines whether or not disappearance of the white reaction has been detected (step S114). In a case where the disappearance of the white reaction has not been detected (step S114: No), the CPU 2 determines whether or not it is a time for automatic image capturing (step S115). In a case where it is not a time for the automatic image capturing (step S115: No), the CPU 2 determines whether or not there is a change in the white reaction, that is, whether or not color of the epithelium of the target region has changed to white (yellow) (step S116). The change in the white reaction is determined based on increase or decrease in area of the white (yellow) part and change in color tone, such as shading. A determination result in step S116 is made use of as a time for image capturing of the target region, as described later. In this processing, it may be configured such that the change in the white reaction is determined based on whether or not a difference in change between a state of the white reaction at the time of last determination and a state of the white reaction at the time of current determination is greater than or equal to a predetermined value and the automatic image capturing is performed every time the difference is greater than or equal to the predetermined value. Alternatively, it may be configured such that the change in the white reaction is determined based on whether or not the amount of change between the state of the white reaction before the white reaction occurs and the state of the white reaction at the time of the current determination is greater than or equal to a predetermined value and the automatic image capturing is performed at every predetermined period of time while the amount of change is greater than or equal to the predetermined value. In addition, the above-described determination method and conditions such as the difference in change, the amount of change, the predetermined value, and the predetermined period of time may be configured to be settable through operation by the operator or the like. The above-described determination is made since the stop operation, the detection of disappearance of the white reaction, and the automatic image capturing are generally not performed immediately after the start of measurement. Details of processing in steps S113 to S115 are described later.

During the colposcopy, a period of time after acetic acid is applied to the target region until a white reaction appears and disappears is observed. Generally, appearance time and disappearance time of a white reaction vary depending on each lesion. In a case of mild findings, the white reaction appears early and disappears early. On the other hand, in a case of severe findings, the white reaction requires time to appear as well as time to disappear. Although a period of approximately 30 to 60 seconds after application of acetic acid is a period when findings are clearest and is suitable for observation, the more malignant a tumor becomes, the longer findings persist, and the observation sometimes takes 3 minutes or longer. In a case where in step S116, no white reaction has appeared (step S116: No), the process returns to step S113 and the processing in steps S113 to S116 is repeated until a white reaction appears.

In a case where a change in color to white (yellow) begins to appear in the epithelium of the target region after the measurement of elapsed time after the acetic acid treatment is started in step S112 and the appearance of the white reaction is detected (step S116: Yes), the CPU 2 calculates a required time after the acetic acid treatment until first appearance of the white reaction and records the calculated required time in the storage 4 (step S117). Next, the notifier 8 notifies the operator or the like of the appearance of the white reaction by emitting an alarm sound (step S118). Next, to capture an image of a portion where the white reaction has appeared, the CPU 2 instructs, as the image capturing condition control, the image capturer 5 to perform focus control to adjust the focus on the detected portion, and the image capturer 5 adjusts the focus on the portion where the reaction has occurred (step S119). In a case where the focus is adjusted on the portion where the white reaction has appeared, the CPU 2 instructs the image capturer 5 to capture an image in which the white reaction has appeared, as image recording control. The image capturer 5 performs image capturing, using the automatic shutter (step S120). At the time of image capturing, the CPU 2 instructs the light emitter 6 to switch the illuminating light, as the image capturing condition control. The light emitter 6 activates the normal light emitting light and the polarized light emitting light in a sequential manner and irradiates the target region with the normal light and the polarized light, and the image capturer 5 captures an image every time each of the normal light and the polarized light is radiated. Because of this configuration, the image capturer 5 captures images of the target region immediately after the white reaction. In a case where the image capturing is performed, the image capturer 5 records a time at which the image capturing is performed (step S121). 28 In a case where the image capturing and the recording of the image capturing time is finished, whether or not the magnitude of change in the color tone due to the white reaction has reached a maximum value, that is, whether or not the white reaction has peaked and the color tone of the portion where the white reaction has appeared is most intense and clearest is determined (step S122). At the beginning of appearance of the white reaction, since area of a portion where the white reaction has appeared gradually expands and the color tone also gradually changes, it is not determined that the white reaction has peaked (step S122: No), and the process returns to step S113 and the processing in and after step S113 is executed again. While the white reaction continues, the stop operation by the operator or the like is generally not performed in step S113, and disappearance of the white reaction is not detected in step S114. In a case where in step S115, an automatic image capturing time is reached (step S115: Yes), the image capturer 5 automatically captures an image of the target region (step S120). The automatic image capturing executed in this step is executed every time the elapsed time after the acetic acid treatment is applied in step S112 reaches a predetermined time. For example, the automatic image capturing is performed a predetermined number of times during a period from when, after the acetic acid treatment is applied, a white reaction starts to appear until the white reaction disappears. Since as described afore, the period of approximately 30 to 60 seconds after the application of acetic acid is a period when findings are clearest and is suitable for observation, the image capturing is performed every 10 seconds during a period of 30 to 90 seconds after applying acetic acid, which includes the above-described period, and further, since the observation requires 3 minutes or longer in some cases, the image capturing is performed every 30 seconds during a period of 90 to 300 seconds, which includes the observation time. As a result, in consideration of a case where an appearance of a white reaction, a change in color tone, or a disappearance of the white reaction cannot be detected and the automatic image capturing cannot record such events, the image capturing is configured to be performed at the above-described preset time intervals to prevent omission of recording.

Since in a case where the white reaction continues and the automatic image capturing time has not been reached in step S115, the white reaction has changed in step S116, the image capturer 5 adjusts the focus on a portion where the white reaction is present, the automatic image capturing is performed, and an image capturing time is recorded in the storage 4 (steps S119, S120, and S121). Note that since the time until the first appearance of the white reaction has already been recorded, the processing in steps S117 and S118 is not executed. As described above, while the white reaction continues, the image capturing of the target region is performed repeatedly.

As the white reaction in the target region proceeds and reaches a peak, the white reaction starts to change in a direction of disappearance. In step S122, the CPU 2 determines, based on detection of this change, that the immediately preceding intensity of the color tone of the white reaction portion is at a maximum (step S122: Yes), and records a position and a shape of the portion where the white reaction is present in the storage 4 (step S123). As a result, an image of the white reaction at its peak with clear findings is recorded without omission.

Although after the peak of the white reaction, the white reaction changes in the direction of disappearance as described before, the image capturing of the target region is repeatedly performed while the white reaction continues. In a case where the white reaction diminishes and the operator or the like determines that the white reaction has disappeared and terminates the observation, the operator or the like performs the stop operation. The stop operation is executed by operating a stop button provided on the camera body or by touching a stop button displayed on the display screen of display 7. Alternatively, it may be configured such that by the operator or the like uttering a voice instructing a stop operation, the voice inputter 10 detects the voice and generates an operation signal, and the CPU 2 executes the stop operation. In a case where the CPU 2 determines that the stop operation is performed by the operator or the like (step S113: Yes), the CPU 2 records time from appearance to disappearance of the white reaction (step S124). In addition, in a case where the stop operation is not performed by the operator or the like, the CPU 2, by detecting disappearance of the white reaction (step S114: Yes), terminates the image capturing processing of the white reaction and records time from appearance to disappearance of the white reaction (step S124). In a case where the recording of the time from the appearance to the disappearance of the white reaction is finished, the CPU 2 terminates the measurement of the elapsed time after the acetic acid treatment (step S125), and the display screen of the display device is switched from the dual screen display of the image of the target region before the white reaction and the live view image input to the image capturer 5 to the single screen display of only the live view image (step S126). After the screen display is returned to the original state, the acetic acid treatment mode is automatically turned off (step S127), and the acetic acid treatment mode is terminated. As described above, in the acetic acid treatment mode, it is possible to assist the operator or the like in such a way as to reduce a burden of operation on the operator or the like, and since a necessary image is saved at an appropriate time, it is also possible to reduce a storage capacity necessary for saving.

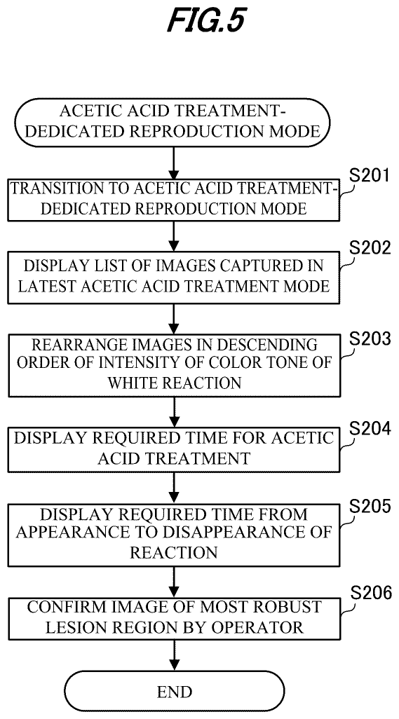

As described above, a plurality of images is captured with respect to a target region by the acetic acid treatment mode. In a case where the image capturing is finished, the captured images are reproduced for confirmation of the captured images. In a normal reproduction mode, the reproduction is performed in order of image capturing. In the present embodiment, in addition to the above configuration, the acetic acid treatment-dedicated reproduction mode that enables a most robust lesion region to be easily identified from a plurality of images captured in the acetic acid treatment mode is provided as a reproduction function. The acetic acid treatment-dedicated reproduction mode is a function that reproduces images in accordance with intensity of color tone of a white reaction portion, and assists the operator or the like in extraction of an image indicating the most robust lesion region. Next, with reference to a flowchart in FIG. 5, the acetic acid treatment-dedicated reproduction mode is described.

In the reproduction of images, first, the operator or the like operates the camera and causes the medical assistance device 1 to transition to the “acetic acid treatment-dedicated reproduction mode” (step S201). In this step, the “acetic acid treatment-dedicated reproduction mode” is turned on by pressing a button on the camera body or by touching the screen on the display device, and a screen for the acetic acid treatment-dedicated reproduction mode is displayed.

In a case where the medical assistance device 1 is caused to transition to the “acetic acid treatment-dedicated reproduction mode”, a list of images that are captured in the latest acetic acid treatment mode among images captured in the afore-described acetic acid treatment mode is displayed on the screen of the display device (step S202). In this step, the list of images is displayed in order of image capturing time. Next, the CPU 2 instructs the display 7 to perform display control to display the images according to change in the white reaction. The display 7 rearranges the images, which are arranged in order of image capturing time, in descending order of intensity of color tone according to shading of white reaction portions, and reproduces and displays the images in the rearranged order (step S203). In this step, the plurality of images to be rearranged is images that are automatically captured in association with detection of change in the white reaction in step S116 in FIG. 4 in the acetic acid treatment mode. Note that a plurality of images automatically captured at a preset time interval may also be rearranged and displayed in an automated manner in descending order of the intensity of the color tone of the white reaction. Note, however, that the above-described images are stored in a separate folder from the images automatically captured in association with the detection of change in the white reaction.

At the time of reproduction of images, the display 7 additionally displays required time for the acetic acid treatment on the screen (step S204). The required time for the acetic acid treatment is time from when acetic acid is applied to the target region until appearance of a white reaction, and the time recorded in step S117 in FIG. 4 in the acetic acid treatment mode is used for the required time. In addition, the display 7 also additionally displays on the screen required time from appearance to disappearance of the white reaction (step S205). As for the required time from the appearance to the disappearance of the white reaction, the time recorded in step S124 in FIG. 4 in the acetic acid treatment mode is used. Since appearance time and disappearance time differ depending on a lesion, the required time serves as a reference for extraction of the most robust lesion region and serves as a criterion for determination of findings. The operator or the like verifies the above-described reproduced images and presses a “confirmation button” of a “most robust lesion” with respect to an image most appropriate as an image confirming the most robust lesion region, and the image of the most robust lesion region is thereby fixed (step S206). Through this processing, time to confirm the most robust lesion region (biopsy region) can be reduced at the time of confirmation by the operator or the like during the acetic acid treatment procedure and recording after biopsy.

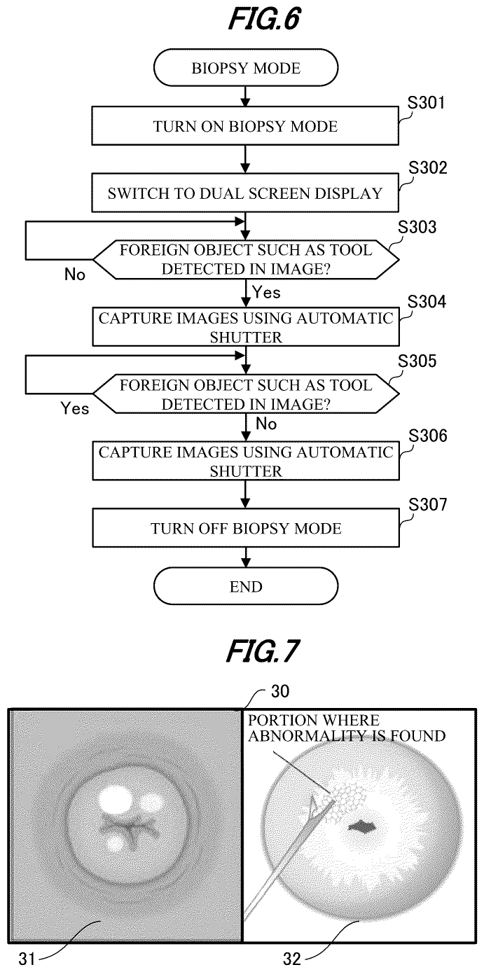

After the image of the most robust lesion region is extracted using the acetic acid treatment-dedicated reproduction mode as described above, a directed biopsy in which an examination target is directed to a portion where an abnormality is detected and tissue is collected from the anomalous portion is performed with the most robust lesion region regarded as a biopsy region. On this occasion, by using the function of the biopsy mode, the operator can perform the directed biopsy while comparing, with the extracted image of the most robust lesion region, and confirming the biopsy region. Next, with reference to a flowchart in FIG. 6, the biopsy mode is described.

At the time of performing a biopsy, first, the operator or the like operates the camera and causes the medical assistance device 1 to transition to the “biopsy mode” (step S301). In this step, the “biopsy mode” is turned on by pressing a button on the camera body or by touching the screen on the display device, and a screen for the biopsy mode is displayed.

After the medical assistance device 1 is caused to transition to the biopsy mode, the display 7 switches the display screen of the display device from the single screen display that displays only a live view image input from the image capturer 5 to a dual screen display that is obtained by dividing the screen into halves and adding an image of the most robust lesion region confirmed in the acetic acid treatment-dedicated reproduction mode (step S302). In FIG. 7, an example of the screen of the display device on which the dual screens are displayed is illustrated. As illustrated in FIG. 7, a screen 30 is divided into halves on the right and left sides, and an image of the most robust lesion region confirmed in the acetic acid treatment-dedicated reproduction mode is displayed on the left-hand side screen 31 and a live view image during biopsy is displayed on the right-hand side screen 32. By displaying the image of the most robust lesion region and the live view image during biopsy side by side, the operator or the like can, while confirming the most robust lesion region, perform the biopsy.

The image processor of the image capturer 5 performs image processing on the image of the biopsy region input from the image capturer 5 and determines whether or not a foreign object other than the biopsy region has been detected (step S303). As used herein, the foreign object refers to a tool to be used in the biopsy, such as forceps, tweezers, a swab, and a syringe, or the like that is different from the uterine cervix, which is the target region. In a case where such a foreign object is captured in the image, it is indicated that the biopsy is being performed. Therefore, in a case where a foreign object other than the biopsy region is determined to be detected (step S303: Yes), the image capturer 5 performs image capturing, using the automatic shutter to record images of the biopsy region during the biopsy (step S304). At the time of image capturing, the CPU 2 instructs the light emitter 6 to switch the illuminating light as the image capturing condition control. The light emitter 6 activates the normal light emitting light and the polarized light emitting light in a sequential manner and irradiates the target region with the normal light and the polarized light, and the image capturer 5 captures an image every time each of the normal light and the polarized light is radiated. In contrast, in a case where such a foreign object is not captured in the image, it is indicated that the process is in a state before the biopsy is performed. Therefore, in a case where a foreign object other than the biopsy region is not determined to be detected (step S303: No), the processing in step S303 is repeated until it is determined that a foreign object is detected and the biopsy is being performed. In a case where the automatic image capturing is performed in step S304, processing of determining whether or not a foreign object has been detected is performed again (step S305). In a case where in this step, no foreign object is detected (step S305: No), it is determined that the tool or the like used in the biopsy is removed and the biopsy is terminated, and to record an image of the biopsy region after biopsy, the image capturer 5 performs image capturing, using the automatic shutter (step S306). In contrast, in a case where the foreign object is continuously detected (step S305: Yes), it is indicated that the biopsy is still being performed, and the processing in step S305 is repeated until it is determined that no foreign object is detected and the biopsy is terminated. In a case where the automatic image capturing is terminated in step S306, the biopsy mode is automatically turned off (step S307), and the biopsy mode is terminated. Note that the biopsy mode may be turned off by the operator or the like operating a button or the like. Since as described above, by using the biopsy mode, the biopsy region is automatically captured at an appropriate time, the operator can save an accurate record of the biopsy region, and the captured records are useful for validity verification of the biopsy region from which tissue is collected. In addition, it becomes possible for the operator, who wears gloves for infection prevention to come into contact with a living body, to perform automatic image capturing without touching the camera during the biopsy procedure, based on a detection of the tool or the like used in the biopsy.

In the above-described embodiment, the acetic acid treatment mode is provided, and at the time of observation of the acetic acid treatment, the medical assistance device 1 measures a required time after the acetic acid treatment until appearance of a white reaction and a required time from the appearance to disappearance of the white reaction, displays the measured required times on the screen of the display device, and records the required times, and, during a period from the appearance to the disappearance of the white reaction, also automatically captures still images of the target region, including the most robust lesion, multiple times and records the still images in conjunction with image capturing times. In contrast, in Embodiment 2, a video of a target region is captured at the time of observation of acetic acid treatment, and after the end of the observation, image extraction processing of extracting, from the video, and recording the above-described required times and a plurality of images during a period from appearance to disappearance of a white reaction is performed.

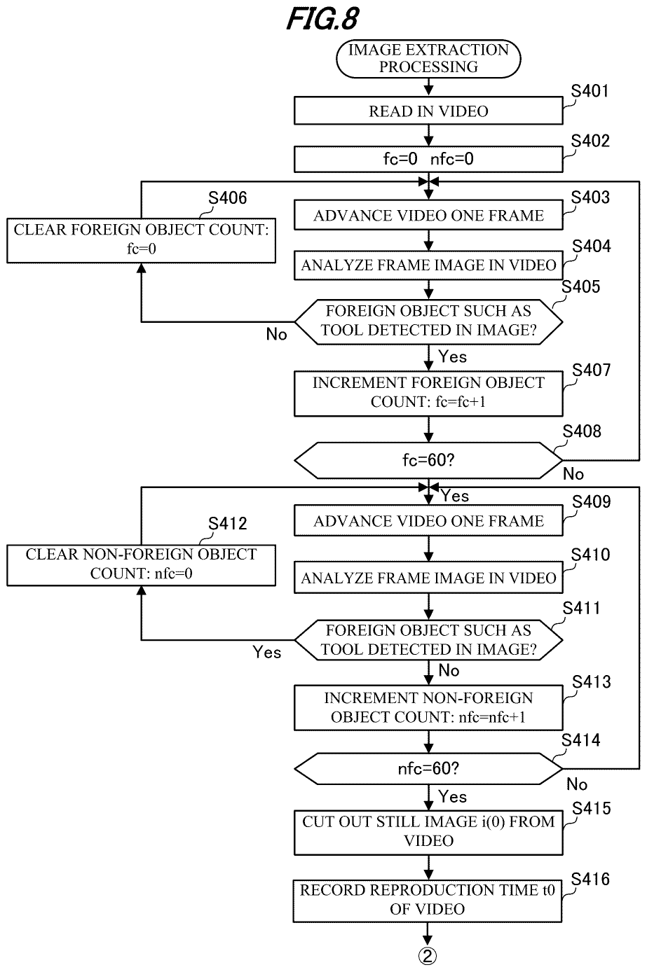

FIGS. 8 to 10 are flowcharts illustrating a control process by which a CPU 2 executes the image extraction processing. FIG. 8 illustrates processing of detecting acetic acid treatment, and processing in steps S403 to S408 indicates processing of detecting a start of acetic acid application and processing in steps S409 to S414 indicates processing of detecting termination of the acetic acid application.

First, the video of the target region captured by a medical assistance device 1 at the time of observation of the target region before and after the acetic acid treatment is read in (step S401). After the video is read in, a count value fc of a foreign object counter that counts up the number of frame images in which a foreign object is detected and a count value nfc of a non-foreign object counter that counts up the number of frame images in which no foreign object is detected are both reset to 0 (step S402). Next, the processing of detecting the start of the acetic acid application is executed. First, the video is advanced one frame (step S403). Next, image analysis is performed on the frame image (step S404), and a foreign object is detected by performing color extraction from the image, performing edge detection with respect to an extracted color region, and extracting a contour shape that is geometric information of the color region. The CPU 2 determines whether or not a foreign object is detected by the image analysis (step S405). In a case where no foreign object is detected (step S405: No), the CPU 2 clears the count value fc of the foreign object counter to 0 (step S406), proceeds to step S403, performs image analysis on the next frame and thereby performs foreign object detection, and determines whether or not a foreign object is detected. The CPU 2 repeats the processing in steps S403 to S406 until a foreign object is detected. In a case where a foreign object is detected, the CPU 2 increments the count value fc of the foreign object counter by +1 (step S407), and next determines whether or not the count value fc has reached a set value “60” (step S408). Herein, “60” is the number of frames and indicates 1 second in a case where a frame rate is 60 fps and 2 seconds in a case where the frame rate is 30 fps. That is, the count value fc indicates elapsed time, and the CPU 2 determines whether or not time during which the foreign object is continuously detected has reached a set time. In a case where the count value fc has not reached 60 (step S408: No), the process returns to step S403, and the processing in and after step S403 is repeated. In a case where in step S408, the count value fc has reached 60 (step S408: Yes), the CPU 2 determines that the foreign object is continuously detected within the set time, that is, processing of applying acetic acid treatment to a target region, using a tool or the like used for the acetic acid treatment is started. Note that although the set value can be arbitrarily set by an operator or the like in consideration of the frame rate of the video, it is necessary to set a value corresponding to a time during which the foreign object is continuously detected to the extent that the acetic acid application is observed to be started.

In a case where the start of the acetic acid application is detected in step S408, the CPU 2 next executes processing of detecting the termination of the acetic acid application in steps S409 to S414. In step S409, the CPU 2 advances the video one frame, performs image analysis on the image (step S410), and determines whether or not the foreign object is still detected, by the image analysis (step S411). In a case where the foreign object is continuously detected (step S411: Yes), the count value nfc of the non-foreign object counter is cleared to 0 (Step S412), the process returns to Step S409, and the processing in steps S409 to S412 is repeated until the foreign object becomes undetectable. In a case where the foreign object becomes undetectable, the count value nfc of the non-foreign object counter is incremented by +1 (step S413), and whether or not the count value nfc has reached a set value “60” is determined (step S414). As with the above description, the set value is the number of frames and indicates the elapsed time, and the CPU 2 determines whether or not the foreign object has not been detected continuously within the time set in this step. In a case where the count value nfc has not reached 60 (step S414: No), the process returns to step S409, and the processing in and after step S409 is repeated. In a case where the count value nfc has reached 60 (step S414: Yes), the CPU 2 determines that the foreign object has not been detected continuously within the set time, that is, the processing of applying the acetic acid treatment to the target region, using a tool or the like used for the acetic acid treatment has been terminated. Note that although the set value can be arbitrarily set by the operator or the like in consideration of the frame rate of the video, it is necessary to set a value corresponding to a time during which the foreign object is not detected continuously to the extent that the acetic acid application is observed to be terminated.

In a case where it is determined that the acetic acid application is terminated, the CPU 2 cuts out a still image i(0) that is an image on which the image analysis is performed at the time

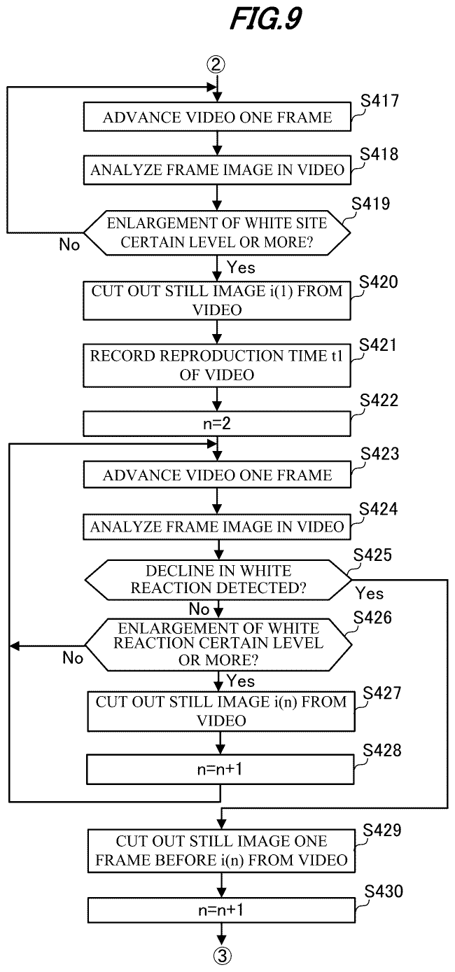

of the determination, from the video (step S415) and displays and records the cut-out still image i(0), and also records reproduction time t0 of the video up until the still image i(0) (step S416). 45 FIG. 9 illustrates the image extraction processing performed during a period from appearance of a white reaction until detection of a most robust lesion, and processing in steps S417 to S421 indicates the image extraction processing at the time of the appearance of the white reaction and processing in steps S423 to S429 indicates the image extraction processing after the appearance of the white reaction until the detection of the most robust lesion. In step S417, the video is advanced one frame, and in step S418, the image analysis is performed on the frame image. The CPU 2 determines, with respect to change in an acetic acid treatment region detected by the image analysis, whether or not enlargement of a white (yellow) site in the region is greater than or equal to a certain level (step S419). The white reaction is detected based on change in color density and change in area of the white (yellow) site. In this processing, the change in the white (yellow) site is determined based on whether or not the amount of enlargement of the white (yellow) site with respect to a state of the acetic acid treatment region in the image at the time of the termination of the acetic acid treatment cut out in step S415 is greater than or equal to a predetermined value. Alternatively, the change in the white (yellow) site may be determined based on whether or not the amount of enlargement of the white (yellow) site with respect to an image one frame or several frames before is greater than or equal to a predetermined value. In a case where the white (yellow) site enlarges, that is, the white (yellow) color becomes deep and the area of the white (yellow) site increases, and the amount of enlargement is not greater than or equal to a predetermined level (step S419: No), the CPU 2 returns to step S417 and execute the processing in and after step S417, and the reproduction of the video is continued until the enlargement of the white (yellow) site reaches a certain level or more. In a case where the amount of enlargement of the white (yellow) site is greater than or equal to the predetermined certain level (step S419: Yes), the CPU 2 determines that a white reaction has appeared and cuts out the frame image from the video as a still image i(1) at the time of appearance of the white reaction (step S420), displays and records the still image i(1), and also records reproduction time t1 of the video up until the still image i(1) (step S421).

In a case where the still image i(1) at the time of appearance of the white reaction is extracted, the CPU 2 sets a variable n to 2. In this processing, the variable n denotes an n-th still image i(n) that is cut out after the appearance of the white reaction. Next, the image extraction processing after the appearance of the white reaction until the detection of the most robust lesion is performed. In step S423, the video is advanced one frame, and in step S424, the image analysis is performed on the frame image. The CPU 2 determines, with respect to change in the white reaction detected by the image analysis, whether or not decline in the white reaction, in which a direction of change in the white reaction changes from an enlarging direction to a diminishing direction, is detected (step S425), and in a case where the white reaction has not changed in the diminishing direction (step S425: No), the CPU 2 determines whether or not the enlargement of the white reaction is greater than or equal to a certain level (step S426). In this processing, whether or not the enlargement of the white reaction is greater than or equal to a certain level is determined based on whether or not the amount of enlargement of the white (yellow) site with respect to the still image i(1) at the time of the appearance of the white reaction is greater than or equal to a predetermined value. Alternatively, whether or not the enlargement of the white reaction is greater than or equal to a certain level may be determined based on whether or not the amount of enlargement of the white (yellow) site with respect to an image one frame or several frames before is greater than or equal to a predetermined value. In a case where the amount of enlargement of the white reaction is not greater than or equal to the predetermined level (step S426: No), the process returns to step S423, the processing in and after step S423 is executed, and the video is reproduced frame by frame until the enlargement of the white (yellow) site reaches the certain level or more. In a case where the amount of enlargement of the white reaction is greater than or equal to the predetermined certain level (step S426: Yes), the CPU 2 determines that the white reaction continues and cuts out the frame image from the video as a still image i(2) after the appearance of the white reaction (step S427) and displays and records the still image i(2). After the still image i(2) is extracted, the CPU 2 increments the variable n by 1 and changes a value of the variable n to n+1. In this step, the CPU 2 changes the value of the variable n to 3(=2+1). After the variable n is changed, the process returns to step S423 and the processing in and after step S423 is executed, and in a case where the amount of enlargement of the white reaction is greater than or equal to the certain level, the CPU 2 cuts out a still image i(n) and displays and records the cut-out still image i(n), changes the value of the variable n to n+1, and likewise executes the processing in and after step S423. In a case where during the above-described repetitive processing, the amount of enlargement of the white reaction falls below the predetermined level, the white reaction changes in the diminishing direction, and the decline in the white reaction is detected (step S425: Yes), it is determined that a most robust lesion has been detected, a frame image one frame before, which is a frame image immediately before the decline, is cut out as the still image i(n) (step S429) and is displayed and recorded. As a result, an image of the most robust lesion is extracted. After the image of the most robust lesion is extracted, the value of the variable n is changed to n+1 (step S430).

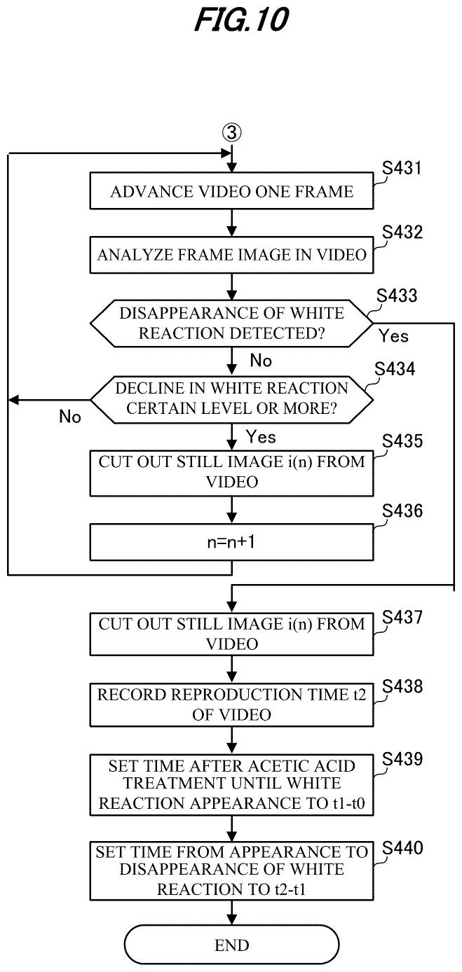

FIG. 10 illustrates the image extraction processing from the decline to the disappearance of the white reaction and calculation processing of required time after the acetic acid treatment until the appearance of the white reaction and required time from the appearance to the disappearance of the white reaction. In step S431, the video is advanced one frame, and in step S432, the image analysis is performed on the frame image. The CPU 2 determines whether or not the disappearance of the white reaction is detected based on the image analysis (step S433). The detection of the disappearance of the white reaction may be determined by the white (yellow) site returning to a state of the acetic acid treatment region in the image at the time of the termination of the acetic acid treatment, or may be determined by the color density and area of the white (yellow) site becoming less than or equal to predetermined levels. In a case where the CPU 2 determines that the disappearance of the white reaction has not been detected (step S433: No), the CPU 2 next determines whether or not the decline in the white reaction is greater than or equal to a certain level (step S434). In this processing, whether or not the decline in the white reaction is greater than or equal to the certain level is determined based on whether or not the amount of decline in the white (yellow) site with respect to the image of the most robust lesion is greater than or equal to a predetermined value. Alternatively, whether or not the decline in the white reaction is greater than or equal to the certain level may also be determined based on whether or not the amount of decline in the white (yellow) site with respect to an image one frame or several frames before is greater than or equal to a predetermined value. In a case where the amount of decline in the white reaction is not greater than or equal to the predetermined certain level (step S434: No), the process returns to step S431 and the processing in and after step S431 is executed, and in a case where the amount of decline in the white reaction is greater than or equal to the predetermined certain level (step S434: Yes), it is determined that the white reaction continues while declining, and the CPU 2 cuts out the frame image from the video as the still image i(n) (step S435), displays and records the still image i(n), and also changes the value of the variable n to n+1 (step S436). After the variable n is changed, the process returns to step S431, and the processing in and after step S431 is executed repeatedly. In a case where in step S433, the disappearance of the white reaction is determined to be detected (step S433: Yes), a disappearance image of the white reaction is cut out as the still image i(n) (step S437), displayed, and recorded in conjunction with reproduction time t2 of the video up until the still image i(n) (step S438). Next, required time t1-t0 after the acetic acid treatment, in which acetic acid is applied to the target region, until the appearance of the white reaction is calculated based on the reproduction time t0 of the video until the end of the acetic acid application and the reproduction time t1 of the video until the appearance of the white reaction, and is recorded (step S439). Further, required time t2-t1 from the appearance to the disappearance of the white reaction is calculated based on the reproduction time t2 of the video until the disappearance of the white reaction, and recorded (step S440). Consequently, the image extraction processing terminates.

Although in Embodiment 2 described above, the medical assistance device 1 is described using the colposcopy camera as an example, the medical assistance device 1 may, without being limited thereto, also be a system that is a combination of an image capturing device such as the colposcopy camera and a viewer device, as described afore. In addition, the medical assistance device 1 may include only the viewer device that can take in a captured image, or may be an information processing device such as a personal computer.

In the above-described embodiments, the CPU 2 functions as a processor by executing the program 12 stored in the storage 4. However, instead of the CPU 2 executing the program 12 stored in the storage 4, the medical assistance device 1 may include dedicated hardware such as an application specific integrated circuit (ASIC), a field-programmable gate array (FPGA), and various types of control circuits, and the dedicated hardware may function as a processor. In this case, a portion of the medical assistance device 1 may be achieved by the dedicated hardware, while the other portion may be achieved by software or firmware. In addition, in the above-described embodiments, the processor may include a single CPU to perform the processing alone or include two or more CPUs to perform processing in collaboration with one another.

In addition, the above-described embodiments may be applied to not only a case where acetic acid is applied to the uterine cervix but also a case where acetic acid is applied to other disease regions such as the ear, nose, and throat and the digestive tract or a case where the application of a chemical liquid other than acetic acid changes a state (color, shape, and the like) of a lesion to a state facilitating observation.

In addition, in the above-described embodiments, not only may the program 12 be stored in the storage 4 in advance, but also the program 12 may be stored in the storage 4 by causing the program 12 to be read in from a computer-readable external recording medium (such as a compact disc (CD)-ROM, a digital versatile disc (DVD)-ROM, a memory card, and a USB memory) via a recording medium reader. In addition to the above, the program 12 can be stored in the storage 4 by superimposing the program 12 on a carrier wave and reading in the program 12 via a communication medium such as the Internet into the storage 4.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. A medical assistance device that assists in performing colposcopy, the medical assistance device comprising
one or more processors configured to:
identify, based on a plurality of images obtained by continuous image capturing, the continuous image capturing continuously capturing images of a target region to which acetic acid treatment is applied, a time at which a predetermined change associated with acetic acid treatment occurs in the target region or an image captured at the time; and
perform, based on the identified time or the identified image, at least one of image capturing condition control in the continuous image capturing, image recording control in the continuous image capturing, and display control of an image recorded by the continuous image capturing.

2. The medical assistance device according to claim 1, wherein
the one or more processors:
identify, during the continuous image capturing, an image in which the predetermined change associated with acetic acid treatment occurs in the target region; and
perform, as the image capturing condition control, focus control to adjust focus on the target region in which the predetermined change occurs, based on the identified image, at a time of image capturing.

3. The medical assistance device according to claim 1, wherein
the one or more processors:
identify, during the continuous image capturing, a time at which the predetermined change associated with acetic acid treatment occurs in the target region; and
perform, as the image capturing condition control, illumination control to switch illuminating light that is radiated on the target region at the identified time, at a time of image capturing.

4. The medical assistance device according to claim 1, wherein
the one or more processors:
identify, during the continuous image capturing, a time at which the predetermined change associated with acetic acid treatment occurs in the target region; and
perform, as the image recording control, recording control to selectively record an image captured at the identified time.

5. The medical assistance device according to claim 1, wherein
the one or more processors:
identify, after recording a plurality of images captured by the continuous image capturing, an image of the target region in which the predetermined change associated with acetic acid treatment occurs, based on the recorded plurality of images; and
performs, as the display control, control to display the identified image.

6. The medical assistance device according to claim 1, wherein
the one or more processors:
acquire change in a white reaction in epithelium of the target region; and
identify, in a case where change in the white reaction is greater than or equal to a predetermined threshold value, a time at which the change in the white reaction is greater than or equal to the predetermined threshold value as an image capturing time of the target region.

7. The medical assistance device according to claim 6, wherein
the white reaction is a reaction in which epithelium of the target region changes whiter or yellower than normal epithelium in surroundings, and
change in the white reaction has a characteristic of being determined by increase or decrease in area of a white or yellow site and change in shading of color tone.

8. The medical assistance device according to claim 6, wherein
the one or more processors
execute automatic image capturing in a case where a difference in change between a state of a white reaction at a time of last determination and a state of a white reaction at a time of a current determination is greater than or equal to the predetermined threshold value.

9. The medical assistance device according to claim 7, wherein
the one or more processors
execute automatic image capturing in a case where a difference in change between a state of a white reaction at a time of last determination and a state of a white reaction at a time of a current determination is greater than or equal to the predetermined threshold value.

10. A medical assistance method, comprising
a medical assistance device that assists in performing colposcopy:
identifying, based on a plurality of images obtained by continuous image capturing, the continuous image capturing continuously capturing images of a target region to which acetic acid treatment is applied, a time at which a predetermined change associated with acetic acid treatment occurs in the target region or an image captured at the time; and
performing, based on the identified time or the identified image, at least one of image capturing condition control in the continuous image capturing, image recording control in the continuous image capturing, and display control of an image recorded by the continuous image capturing.

11. A non-transitory computer-readable recording medium storing a program causing a medical assistance device that assists in performing colposcopy to execute processing comprising:
identifying, based on a plurality of images obtained by continuous image capturing, the continuous image capturing continuously capturing images of a target region to which acetic acid treatment is applied, a time at which a predetermined change associated with acetic acid treatment occurs in the target region or an image captured at the time; and
performing, based on the identified time or the identified image, at least one of image capturing condition control in the continuous image capturing, image recording control in the continuous image capturing, and display control of an image recorded by the continuous image capturing.
